(19) United States
(12) Patent Application Publication
(10) Pub. No.: US 2006/0252831 A1
(43) Pub. Date: Nov. 9, 2006

(54) Method for the treatment of magnesium and potassium deficiencies

(76) Inventors: Christopher Offen, Marietta, GA (US); Stephen F. Brandon, Roanoke, TX (US)

Correspondence Address: Erinn C. Kelly;ARNALL GOLDEN GREGORY LLP, 171 17th Street NW, Atlanta, GA, 30363, US

(21) Appl. No.: 11/123,285

(22) Filed: May 6, 2005

(51) Int. Cl. A61K 31/19

(52) U.S. Cl. 514/557

(57) ABSTRACT

A method for the treatment of depleted intracellular and serum magnesium levels by administration of a highly bioavailable magnesium salt is disclosed. A prescription dispensing system is also disclosed. The highly bioavailable magnesium salt can be administered alone or as adjunctive therapy in conjunction with various medications that cause or exacerbate depleted intracellular magnesium levels, such as renal magnesium wasting medications, including diuretics, immunosuppressants, chemotherapeutic agents, and antibiotics. The highly bioavailable magnesium salt can also be used as adjunctive therapy in conjunction with Class III anti-arrhythmic drugs to attenuate the QTc interval and reduce the risk of fatal arrhythmias, which are a common risk associated with Class III anti-arrhythmic drugs. The administration of a highly bioavailable magnesium salt in accordance with the present invention also serves to restore intracellular potassium levels to normal ranges in patients who remain hypokalemic despite potassium therapy.

### BACKGROUND OF THE INVENTION

1. Field of the Invention

The present invention relates to pharmaceutical formulations and, more specifically, a method for the treatment of depleted intracellular and serum magnesium levels and for the treatment of various conditions relating to magnesium and potassium deficiencies, including arrhythmia and the related potentially fatal condition Torsades de Pointes, through the use of a highly bioavailable oral magnesium salt, both alone and as adjunctive therapy with various medications.

2. Description of the Prior Art

Magnesium is the fourth most abundant cation in the human body, the second in the intracellular environment, and takes part in more than 300 enzymatic reactions. Magnesium is also essential for normal functioning of many of the body's organs, including the heart and kidneys. Magnesium deficiency is associated with an extensive list of diseases and conditions, including heart disease, arrhythmia, diabetes, migraine headaches and osteoporosis. These conditions affect a tremendous number of people in the United States alone.

There are over 5,000,000 patients in the United States who suffer from congestive heart failure. Atrial fibrillation is the most common cardiac tachyarrhythmia in the United States affecting approximately 2 million patients and is associated with substantial increases in patient morbidity, mortality, and healthcare expenditures. Fifteen percent of all cerebrovascular accidents are caused by atrial fibrillation.

Unfortunately, the medications that are prescribed to treat these conditions often cause or exacerbate magnesium depletion or other life-threatening conditions. Standard therapy for congestive heart failure patients includes loop or thiazide diuretics and digitalis. Loop or thiazide diuretics significantly deplete magnesium, as well as potassium, as adequate magnesium levels are necessary for the proper maintenance of potassium balance.

When a patient's heart rhythm becomes irregular, cardioversion is frequently employed to restore normal sinus rhythm. Although patients are more likely to convert to normal sinus rhythm with electric current cardioversion, pharmacologic cardioversion is often employed as a first-line strategy due to patient fears and the inherent risks associated with electric current cardioversion and associated anesthesia. The most common agents used for pharmacologic cardioversion include sotalol, dofetilide, and ibutilide. Dofetilide and sotalol are Vaughn-Williams Class III anti-arrhythmic agents that inhibit the rapid component of the delayed potassium rectifier channel, resulting in a prolongation of the ventricular action potential duration. This is detected clinically as a prolongation of the QTc interval on the electrocardiogram (ECG) thus increasing the risk for the development of Torsade de Pointes (TdP). Sotalol and dofetilide demonstrate dose-dependent prolongation of the QTc interval and incidence of TdP. The risk of Torsade de Pointes is greatest soon after initiation of the drug, or with major increases in dosage. Some patient groups are at higher risk of this complication, such as patients with histories of congestive heart failure, patients on other anti-arrhythmic drugs, or patients with particularly slow heart rates, depleted electrolytes, and other predisposing factors. These safety concerns require hospitalization for drug initiation, so that prompt resuscitation can be performed should a patient develop a ventricular arrhythmia from the drug. This limits the use of sotalol and dofetilide, thus preventing eligible patients from obtaining the morbidity and/or mortality benefits associated with their use. Class Ia anti-arrhythmic agents quinidine and procainamide prolong the QTc interval by the same mechanism. Other drugs that are known to prolong the QTc interval have been found in most other classes of therapeutic agents, including anti-histamines, antibiotics such as moxifloxacin and erythromycin, gastrointestinal prokinetics, and anti-schizophrenia medications such as ziprasidone.

Further, while it is generally known that magnesium is an important nutrient and that people should ideally take a daily supplement in order to maintain normal levels of magnesium in the body, a medical magnesium deficiency should be diagnosed and treated by a doctor, and a doctor should monitor the patient's treatment. This is because not all magnesium supplements are the same; if the magnesium salt used in the supplement has a low bioavailability, very little if any magnesium will actually be absorbed into the body and reach the cells where the magnesium is needed. Such magnesium salts that have low bioavailabilities include magnesium oxide, magnesium hydroxide, magnesium chloride, magnesium sulfate and magnesium gluconate. Because over the counter magnesium supplements do not provide the consumer with information regarding the bioavailability of the vitamins and minerals that they include, it is very difficult for consumers to know whether the supplement they are taking is actually repleting their magnesium deficiency. Ingesting too much of a magnesium salt with low bioavailability can induce diarrhea, which is potassium wasting.

It is also possible that the consumer, if not monitored by a physician, could take a magnesium supplement that uses a magnesium salt having a high bioavailability, resulting in the consumer ingesting too much magnesium. While magnesium is generally safe, due to the physiological relationship between magnesium and potassium, taking a magnesium supplement having a high magnesium bioavailability will result in not only restoring intracellular and serum magnesium levels, but also restoring potassium levels. Extremely high intracellular potassium levels are toxic. Therefore, it is imperative that patients taking a highly bioavailable magnesium salt be monitored by a physician.

It is known to use magnesium to treat conditions that have a connection to hypomagnesaemia. United States and European clinical trials have demonstrated that about 40% of migraine patients have low brain magnesium levels. In a number of controlled trials, oral magnesium has been shown to reduce the frequency and severity of migraine attacks, particularly in women. European trials clearly show that magnesium is as important as calcium in preventing and treating osteoporosis and United States physicians have started to recognize this as well. It also has been established in one trial that intravenous magnesium attenuates the increase in QTc intervals in patients receiving ibutilide. In other trials, intravenous magnesium sulfate has been shown to be an effective antiarrhythmic agent and an efficacious adjunctive therapy as well. Two small randomized trials comparing intravenous magnesium sulfate to either verapamil or diltiazem found an enhanced conversion rates (58% vs 23%, p=0.01 and 57% vs 22%, p=0.03, respectively) with magnesium. However, intravenous treatment restricts patients to in-patient hospital care.

Further, magnesium salts currently used to treat magnesium deficiencies, such as magnesium oxide, magnesium chloride, magnesium sulfate, magnesium gluconate or magnesium hydroxide, all of which exhibit extremely low to moderate bioavailability, from about 2% for magnesium oxide to about 20% for magnesium chloride and magnesium gluconate. Two 6month studies of patients with atrial fibrillation found oral magnesium hydroxide to be ineffective at maintaining sinus rhythm.

To date, trials with existing oral magnesium formulations have not established the ability of oral magnesium to attenuate the QTc interval. Further, no placebo controlled trial has shown the ability of a magnesium supplement to achieve normal intracellular or serum levels. This is because the oral magnesium products evaluated to date are of low bioavailability and poorly absorbed. The low bioavailability of most magnesium supplements also means that higher doses are required to try to achieve the desired result. Low bioavailability and poor absorption also commonly lead to diarrhea and nausea. Therefore, it is often not possible for patients to ingest the levels of magnesium that would be required to achieve normal intracellular or serum magnesium levels.

Further, the serum magnesium level is not a reliable way for determining total body magnesium depletion because of the minimal extracellular concentration. If the serum level is low, there is clearly a deficiency. However, intracellular magnesium levels may be markedly depleted before the serum level drops. For example, there is no correlation between intracellular atrial magnesium and serum magnesium. One study found that intracellular magnesium concentrations went down in mongrel dogs as they developed pacing induced heart failure. Mongrel myocytes dialyzed with lower magnesium concentrations had a longer repolarization time than those mycocytes given higher magnesium concentrations.

As such, it is desirable to have a highly bioavailable form of magnesium that can be prescribed by physicians in dosages necessary to achieve normal intracellular magnesium levels, which are defined as a minimum concentration of 33.9 mEq/IU, as well restoring intracellular potassium levels. It is also desirable to have a highly bioavailable form of magnesium that can work in conjunction with Class III anti-arrhythmic medications and other IKr potassium channel blockers to attenuate the QTc interval, thereby minimizing the risk of the occurrence of Torsade de Pointes. It is also desirable to have a highly bioavailable form of magnesium that can effectively counter the renal magnesium wasting caused by certain medications. It is also desirable to have an oral dosage form of a sustained release magnesium supplement that would allow patients to take an oral magnesium product on an out-patient basis, thus eliminating the need for extensive hospital stays for the purpose of monitoring the patient for the possible occurrence of life-threatening arrhythmias. It is also desirable for the dosage of the anti-arrhythmia medication to be lowered, also minimizing the risk of development of Torsades de Pointes and possibly eliminating the necessity for admittance for continuous observation. It is also desirable to have a highly bioavailable form of magnesium that allows the anti-arrhythmic drug to be used at higher dose with similar side effects or a lower dose with similar efficacy. It is also desirable to have a highly bioavailable magnesium salt that serves to restore intracellular potassium levels to normal ranges in patients who remain hypokalemic despite potassium therapy. It is also desirable to have a highly bioavailable form of magnesium that can be co-packaged and/or co-formulated with other medications for the convenience of the physician and the patient.

### SUMMARY OF THE INVENTION

The present invention includes a method for restoring depleted intracellular and serum magnesium levels to normal ranges by administering a highly bioavailable magnesium salt, such as magnesium l-lactate dihydrate, to a patient, either alone or as adjunctive therapy with medications that cause renal magnesium wasting, including diuretics, immunosuppressants, chemotherapeutic agents, and antibiotics.

The present invention also includes a method of attenuating the QTc interval to acceptable levels by administering a highly bioavailable magnesium salt, such as magnesium l-lactate dihydrate, in conjunction with Class III and Class Ia anti-arrhythmic drugs or other medications that prolong the QTc interval, such as anti-schizophrenic medications and antibiotics or other IKr potassium channel blockers, thereby greatly reducing the risk of the occurrence of Torsade de Pointes.

In the present invention, the highly bioavailable form of magnesium can help to greatly reduce the risk of life-threatening arrhythmias by being prescribed for patients who have been implanted with an implantable cardioversion device, the magnesium functioning to replete intracellular magnesium levels, leading to fewer arrhythmic episodes.

In accordance with the present invention, studies have demonstrated that magnesium lactate is 100% effective in repleting intracellular magnesium levels to normal ranges, which is defined as a minimum concentration of 33.9 mEq/IU and can be measured by electron fluoroscopy or other scientifically acceptable means of measuring intracellular magnesium levels.

The present invention also provides a method of restoring intracellular potassium levels to normal ranges in patients who remain hypokalemic despite potassium therapy by the administration of a highly bioavailable magnesium salt.

These and other aspects of the invention will become apparent from the following description of the preferred embodiments. As would be clear to one skilled in the art, many variations and modifications of the invention may be effected without departing from the spirit and scope of the disclosure.

### DETAILED DESCRIPTION OF THE INVENTION

A preferred embodiment of the invention is now described in detail. As used in the description herein and throughout the claims, the following terms take the meanings explicitly associated herein, unless the context clearly dictates otherwise: the meaning of “a,” “an,” and “the” includes plural reference, the meaning of “in” includes “in” and “on.”

The present invention provides for the use of highly bioavailable form of a magnesium salt, alone or in conjunction with other medications, for the treatment of depleted intracellular and serum magnesium levels. The highly bioavailable form of magnesium salt preferably has a bioavailability of about 40%. Examples of such magnesium salts include: magnesium lactate, magnesium dl-aspartate and magnesium l-aspartate. Preferably, the magnesium salt that is used is magnesium l-lactate dihydrate, which is sold in a sustained release tablet form under the name MagTab SR®. One illustrative formulation of this sustained release tablet form of magnesium l-lactate dihydrate is disclosed in detail in U.S. Pat. No. 5,002,774, which is incorporated herein by reference.

The present invention includes a method of reducing the prolonged QTc interval in arrhythmia patients caused by Class III anti-arrhythmic medications, as well as a method of treating depleted intracellular magnesium levels caused by renal magnesium wasting medications. Typical dosage forms and therapy ranges for Class III anti-arrhythmic drugs, utilized individually are as follows:

| Name of Product | Current Dosage Form | Dosage Range Per Day |
|---|---|---|
| Amiodarone | 200 mg tablet | 200-1600 mg |
| Amiodarone | IV - 50 mg per ml | 150-1000 mg |
| | (3 ml vial) | |
| Dofetilide | 150 mcg | 125-1000 mcg |
| | 250 mcg | |
| | 500 mcg | |
| Sotalol | 80 mg | 80-600 mg |
| | 120 mg | |
| | 160 mg | |
| | 240 mg | |
| Ibutilide Fumerate | IV 1 mg/10 ml vial | 1 or 2 .5 mg infusions |
| | | (0.005 mg per kg per dose |

There are other drugs that also prolong the QTc interval. Class Ia anti-arrhythmic agents quinidine and procainamide prolong the QTc interval by the same mechanism as Class III anti-arrhythmic agents. Another example is ziprasidone, a schizophrenia drug, and another is moxifloxacin, an antibiotic. Typical dosage forms and therapy ranges for ziprasidone and moxifloxacin, utilized individually, are as follows:

| | Current | |
|---|---|---|
| Name of Product | Dosage Form | Dosage Range Per Day |
| Ziprasidone mesylate (Geodon) | 20 mg | 40 mg-200 mg |
| | 40 mg | |
| | 60 mg | |
| | 80 mg | |
| Ziprasidone mesylate (Geodon) | IV 20 mg/ml | 10 mg-40 mg |
| Moxifloxacin Hcl (Avelox) | 400 mg | 400 mg |

A randomized, double-blind, placebo-controlled study was performed at Hartford Hospital. People taking dofetilide or sotalol therapy for at least 5 half-lives were eligible for inclusion. People were excluded for the following reasons: (1) hypersensitivity to magnesium L-lactate, (2) use of a magnesium supplement within 48 hours prior to randomization, (3) use of any of the following agents: verapamil, trimethoprim/sulfamethoxazole, procholorperazine, cisapride, negestrol, cimetidine, triamterene, macrolides, flurorquinolones, sodium polystyrene, diltiazem, nefazodone, azole antifungals, quinine, zafirlukast, select serotonin reuptake inhibitors, protease inhibitors, metformin, tricyclic antidepressants, or phenothiazines, or (4) use of amiodarone within 3 months of randomization. After enrollment, people who discontinued their sotalol or dofetilide or altered their dosage of these drugs were excluded as well. Thirty-four participants were enrolled in the study. Three were subsequently excluded because the antiarrhythmics they were using were discontinued or the dose of the antiarrhythmics changed during the study. Given the known baseline differences in QTc intervals among men and women, each gender was randomized separately using stratified allocation. Patients completing the study (n=30) were well matched for baseline characteristics, which are reflected in Table 1. Intracellular concentrations of essential elements were available for 19 subjects. Overall 63.2% of patients (regardless of experimental group) had baseline intracellular magnesium concentrations below the normal reference range of 33.9-41.9 mEq/IU with an average level of 32.6±2.2 mEq/IU. All of the other baseline intracellular elements evaluated (phosphorus, chloride, calcium, potassium, and sodium) were within the reference range although the phosphorus was at the top of the range (average=16.7±2.8 mEq/IU with reference range of 14.2-17.0 mEq/IU).

Participants received three tablets of magnesium L-lactate (Mag-Tab SR®; Niche Pharmaceuticals; Roanoke, Tex.) or matching placebo twice daily (504 mg of elemental magnesium daily) for 48 hours (i.e. t=0, 12 h, 24 h, 36 h, and 48 h). A 12-lead electrocardiogram (ECG) was recorded at 25 mm/sec at rest prior to initiation of treatment (i.e. baseline) and at 3 and 51 hours after the first dose of magnesium (i.e. t=3 h and t=51 h) for each participant. A single blinded investigator read all ECGs using a precision ruler of 0.5 mm scale (Schlaedler-Quinzel, Inc. Parsippany, N.J.).

QT intervals were measured from the onset of the Q wave (or R/S wave if there was no Q wave) to the end of the T wave where it merges with the isoelectric baseline. The beginning and end of the QRS complex were determined by visual inspection, whereas the end of the T-wave was obtained by extrapolating the descending slope of the T-wave to the isoelectric baseline. When a U wave interrupts the down-sloping of the T-wave, the visible portion of the T wave was extrapolated to the T-P baseline to define the end of the T-wave. The R-R interval was measured from the peak of one R wave to the peak of an adjacent R wave. The QTc interval was corrected for heart rate using Bazett's formula [QTc=QT/(RR)^{1/2}].

Sublingual buccal smears were obtained from a subgroup of participants prior to initiation of study therapy and following the third dose of magnesium using EXAtest™ kits (Intracellular Diagnostics, Inc.; Foster City, Calif.). Samples were analyzed by energy-dispersive X-ray Analysis for intracellular concentrations of magnesium, phosphorus, chloride, calcium, potassium, and sodium. The use of buccal cell smears for intracellular magnesium was performed because it is noninvasive, reproducible, and correlates well (r=0.68, p<0.002) with atrial cell intracellular magnesium.

Continuous data was expressed by a mean±SD. A p value less than 0.05 was considered statistically significant. The primary analysis was the intergroup comparisons of the change in QTc interval from baseline at 3 hours and 51 hours using Bazett's formula. Evaluations of the QRS, QT, and RR intervals were compared between groups at each time period as well. Mann-Whitney tests were used for all statistical analyses of continuous ECG data. Intracellular element intragroup comparisons were performed with a paired t-test. Chi² or Fischer's Exact tests were used for categorical comparisons. Statistical analysis was performed using SPSS version 5.

Results:

The ECG interval comparisons are presented in Table 2. QTc interval reductions from baseline were greater in the magnesium group than the placebo group at 3 hours and 51 hours (p=0.015 and p<0.001). The QT interval change from baseline showed greater reductions in the magnesium than placebo groups at 3 hours (p=0.076) and 51 hours (p=0.042).

The changes in QRS and RR intervals from baseline were not different between the magnesium and placebo groups at 3 hours (QRS: p=0.510 and RR: p=0.749) or 51 hours (QRS: p=0.696 and RR: p=0.245), respectively.

After magnesium supplementation the intracellular magnesium concentrations rose significantly (p=0.002) with all patients having baseline intracellular magnesium concentrations below the normal reference range achieving a concentration within the normal reference range. After placebo was given, intracellular magnesium concentrations were unchanged (p=0.320). No changes occurred with the other elements after magnesium or placebo administration (Table 3). In addition to the one participant who withdrew secondary to diarrhea, another participant reported “loose bowels” but this was said to be mild and did not negatively impact the subject.

Diuretics were used in 47% of patients. When the QTc intervals were compared at baseline, 3 hours, and 51 hours between those receiving diuretics and those without diuretics (regardless of study drug randomization), no differences were noted. Further, in the group receiving diuretics, those randomized to magnesium had dramatic reductions in the QTc interval as compared to those receiving placebo at 3 hours (51.1±42 vs 10.7±20.1 msec, p=0.036). At 51 hours, the magnesium group had a 24.7±24.3 msec reduction while the placebo group had a 2.1±12.4 msec increase, p=0.011. In the no diuretic group, the QTc intervals were reduced from baseline by 25.5±27.9 and 26.2±27.4 msec in the magnesium group at 3 and 51 hours and were increased by 3.1±22.2 and 4.6±17.4 msec in the placebo group at 3 and 51 hours (p=0.078 and p=0.027, respectively). Qualitatively, greater reductions in the QTc interval occurred at 3 hours in magnesium treated patients (p=0.220) with diuretic therapy as compared to those without diuretics, but no difference between the magnesium groups occurred at 51 hours (24.7±24.3 vs 26.2±27.4 msec, p=0.919). Baseline intracellular magnesium concentrations were not different among those receiving diuretics or not (33.0±2.2 vs 32.1±2.4, p=0.468).

Discussion

In this study, participants with arrhythmias requiring treatment with sotalol or dofetilide had a baseline intracellular deficiency in magnesium that was not explained by diuretic use. Use of magnesium L-lactate corrected the deficiency at the 51-hour time point. Other essential elements such as sodium, calcium, and potassium did not show tissue abundance or deficiency although intracellular phosphorus averaged towards the top of the normal reference range.

Magnesium L-lactate therapy reduced the QTc intervals by 37 msec at 3 hours post-dosing (corresponding to the expected maximal serum concentrations (Cmax) of the product) and 25 msec at 51 hours. QTc interval reductions with magnesium L-lactate were attenuated at 3 hours among participants receiving diuretics but the effect was transient and by 51 hours, there was no difference in QTc interval reductions germane to diuretic use. Given the quick onset of appreciable QTc interval reductions with magnesium L-lactate, acute use of the oral product in a patient at risk of Torsade de Pointes may obviate the need for intravenous access. The QT interval was homogenously reduced by 15 ms at 3 hours and 14 ms at 51 hours with magnesium therapy.

According to the Long QT Syndrome Registry, there is a direct relationship between the degree of QTc interval prolongation and ventricular arrhythmic events (defined as probable QTc prolongation related syncope or sudden cardiac death) as expressed by the formula [hazard ration=1.052^{X}; where x=the increase in QTc interval in msec]. As such, if a 23 msec chronic suppression of the QTc interval could be achieved, a 3.2 fold reduction in risk would result.

Among the 34 patients in the study, 2 patients did experience loose bowel movements and one withdrew from the study. The patient who withdrew reported having diarrhea 30 minutes after dosing. Based on the osmotic mechanism of magnesium catharsis, it is unlikely that magnesium L-lactate was the cause since the drug would not have been in the large intestine at the time.

### CONCLUSION

Combining magnesium L-lactate with sotalol or dofetilide reduces the QTc interval significantly in the short term. A baseline deficiency in intracellular magnesium but not other essential elements such as calcium, potassium, and sodium suggest that there may be need for chronic magnesium supplementation in this population.

Oral magnesium L-lactate raises intracellular magnesium concentrates from subnormal 32.6±2.2 mEq/IU to normal 36.2±2.4 mEq/IU levels (p=0.002) at steady state and lowers the QTc intervals from 472.5±54.1 at baseline to 435.9±63.6 at 3 hours (p=0.015) and 449.4±51.5 at 51 hours (p<0.001) of patients receiving sotalol or dofetilide. Placebo does not alter intracellular magnesium levels or the QTc interval of patients taking sotalol or dofetilide.

Tables

TABLE 1
Patient Demographic Comparison.

| | Magnesium | | |
|---|---|---|---|
| | (n = 14) | Placebo (n = 16) | P-Value |
| Gender (% Male) | 13 (92.9) | 14 (87.5) | 0.903 |
| Age (years | 66.5 ± 12.5 | 72.1 ± 7.3 | 0.138 |
| Sotalol (%) | 10 (71.4) | 12 (75.0) | 0.847 |
| Dofetilide (%) | 4 (29.6) | 4 (25.0) | 0.847 |
| Hypercholesterolemia (%) | 9 (64.3) | 7 (43.8) | 0.448 |
| Diabetes (%) | 3 (21.4) | 2 (12.5) | 0.642 |
| Coronary Dx (%) | 11 (78.6) | 12 (75.0) | 0.840 |
| Heart Failure (%) | 5 (35.7) | 2 (12.5) | 0.286 |
| AF/AFL Hx (%) | 4 (28.6) | 9 (56.3) | 0.316 |
| VT/VF Hx (%) | 13 92.9) | 11 (68.8) | 0.234 |
| Diuretics (%) | 7 (50.0) | 7 (43.8) | 0.980 |
| ACEI/ARB (%) | 8 (57.1) | 9 (56.3) | 0.749 |
| Beta-Blocker (%) | 8 (57.1) | 6 (37.5) | 0.478 |

ACEI = ACE Inhibitor,
AF = Atrial Fibrillation,
AFL = Atrial Flutter,
ARB = Angiotensin II Receptor Blocker,
Dx = Disease,
Hx = History

TABLE 2
Electrocardiographic Comparison.

| (Units in Msec) | Magnesium (n = 14) | Placebo (n = 16) | P-Value |
|---|---|---|---|
| QRS Interval Baseline | 134.4 ± 419 | 138.4 ± 28.4 | 0.756 |
| QRS Interval 3 Hour | 143.6 ± 41.7 | 131.9 ± 32.5 | 0.510 |
| QRS Interval 51 Hour | 130.3 ± 43.9 | 136.6 ± 27.3 | .0696 |
| QT Interval Baseline | 430.5 ± 38.1 | 446.9 ± 35.6 | 0.240 |
| QT Interval 3 Hour | 415.2 ± 40.1 | 444.6 ± 35.3 | 0.076 |
| QT Interval 51 Hour | 416.7 ± 44.6 | 446.0 ± 35.7 | 0.032 |
| RR Interval Baseline | 848.0 ± 163.9 | 964.5 ± 193.9 | 0.088 |
| RR Interval 3 Hour | 829.5 ± 111.1 | 952.8 ± 144.3 | 0.749 |
| RR Interval 51 Hour | 868.2 ± 117.6 | 943.0 ± 152.5 | 0.245 |
| QTc Baseline | 472.5 ± 54.1 | 459.6 ± 43.8 | 0.667 |
| QTc Interval 3 Hour | 435.9 ± 63.6 | 450.4 ± 47.7 | 0.015 |
| QTc Interval 51 Hour | 449.4 ± 51.5 | 463.1 ± 47.8 | <0.001 |

P-values at baseline were based on intergroup comparisons while p-versions at 3 and 48 hours were based on the change from baseline intergroup comparisons.

TABLE 3
Intracellular Elemental Concentration Comparison.

| | | | |
|---|---|---|---|
| (Units in mEq/IU) | Baseline | Post-Dosing | P-Value |

Magnesium Group (n = 10)

| | | | |
|---|---|---|---|
| Magnesium | 32.2 ± 2.2 | 36.2 ± 2.4 | 0.002 |
| Phosphorus | 15.9 ± 2.2 | 16.5 ± 1.7 | 0.926 |
| Chloride | 4.5 ± 1.4 | 3.9 ± 0.6 | 0.350 |
| Calcium | 4.2 ± 0.4 | 4.3 ± 0.6 | 0.965 |
| Potassium | 119.1 ± 22.2 | 96.7 ± 20.4 | 0.116 |
| Sodium | 4.4 ± 0.8 | 4.1 ± 0.4 | 0.348 |

Placebo Group (n = 9)

| | | | |
|---|---|---|---|
| Magnesium | 33.1 ± 2.2 | 34.1 ± 1.6 | 0.320 |
| Phosphorus | 17.5 ± 3.3 | 16.1 ± 1.5 | 0.112 |
| Chloride | 4.1 ± 1.4 | 3.8 ± 0.6 | 0.450 |
| Calcium | 5.2 ± 2.4 | 3.9 ± 0.4 | 0.199 |
| Potassium | 129.4 ± 46.8 | 113.2 ± 29.6 | 0.246 |
| Sodium | 4.0 ± 0.5 | 3.9 ± 0.4 | 0.252 |

The present invention is also embodied as a prescription dispensing system for attenuating a prolongation of the QTc interval. The prescription dispensing system includes a pharmaceutically effective dose of a highly bioavailable magnesium salt and a pharmaceutically effective dose of a drug, a known side effect of which is a prolongation of the QTc interval. Such drugs include Class Ia and III anti-arrhythmic drugs, antibiotics, and anti-schizophrenic drugs. The highly bioavailable magnesium salt and the drug can be co-formulated as any oral or intravenous form of medication, such as an immediate or sustained release capsule, tablet, ingestible liquid, powder, gel, or intravenous injection, or into a patch. Appropriate co-formulation methods are known in the art. The highly bioavailable magnesium salt and the drug can also be formulated separately, with the highly bioavailable magnesium salt formulated as any form of oral or intravenous medication, such as an immediate or sustained release capsule, tablet, ingestible liquid, powder, gel, or intravenous injection, or into a patch, and then co-packaged into a dispensing container. The dispensing container can be a blister pack, a bottle, or a syringe. Such dispensing containers are known in the art. Further, the prescription dispensing system can contain a single dose, a multiple dose daily regimen, or a multiple day regimen of the highly bioavailable magnesium salt and the drug.

The present invention is also embodied as a prescription dispensing system for treating renal magnesium wasting. The prescription dispensing system includes a pharmaceutically effective dose of a highly bioavailable magnesium salt and a pharmaceutically effective dose of a drug, a known side effect of which is renal magnesium wasting. Such drugs include diuretics, immunosuppressants, and chemotherapeutic drugs. The highly bioavailable magnesium salt and the drug can be co-formulated as any oral or intravenous form of medication, such as an immediate or sustained release capsule, tablet, ingestible liquid, powder, gel, or intravenous injection, or into a patch. Appropriate co-formulation methods are known in the art. The highly bioavailable magnesium salt and the drug can also be formulated separately, with the highly bioavailable magnesium salt formulated as any form of oral or intravenous medication, such as an immediate or sustained release capsule, tablet, ingestible liquid, powder, gel, or intravenous injection, or into a patch, and then co-packaged into a dispensing container. The dispensing container can be a blister pack, a bottle, or a syringe. Such dispensing containers are known in the art. Further, the prescription dispensing system can contain a single dose, a multiple dose daily regimen, or a multiple day regimen of the highly bioavailable magnesium salt and the drug.

The present invention also includes a method for restoring depleted intracellular potassium levels in patients who otherwise remain hypokalemic despite potassium therapy by administering a highly bioavailable magnesium salt.

The above-described embodiments are given as illustrative examples only. It will be readily appreciated that many deviations may be made from the specific embodiments disclosed in this specification without departing from the invention. Accordingly, the scope of the invention is to be determined by the claims below rather than being limited to the specifically described embodiments above.

What is claimed is:

1. A method of restoring depleted intracellular magnesium levels, comprising the step of administering a highly bioavailable magnesium salt in a pharmaceutically acceptable amount sufficient to restore depleted intracellular magnesium levels to a therapeutically acceptable level.

2. The method of claim 1, wherein the highly bioavailable magnesium salt has a bioavailability of at least about 30%.

3. The method of claim 2, wherein the highly bioavailable magnesium salt has a bioavailability of at least about 40%.

4. The method of claim 1, wherein the highly bioavailable magnesium salt comprises magnesium l-lactate dihydrate.

5. The method of claim 1, wherein the highly bioavailable magnesium salt is formulated in a formulation selected from a group consisting essentially of an immediate release tablet, a capsule, a gel, an ingestible liquid, a powder, a patch, and an intravenous injection.

6. The method of claim 1, wherein the highly bioavailable magnesium salt is formulated in a formulation selected from a group consisting essentially of a sustained release tablet, a capsule, a gel, an ingestible liquid, a powder, a patch, and an intravenous injection.

7. The method of claim 1, wherein the highly bioavailable magnesium salt is administered in a dosage of between about 3 mEq/IU and about 60 mEq/IU per day.

8. The method of claim 7, wherein the highly bioavailable magnesium salt is administered in a dosage of about 40 mEq/IU per day.

9. The method of claim 1, wherein the intracellular magnesium levels are restored to a concentration of at least 33.9 mEq/IU.

10. The method of claim 1, wherein depleted intracellular potassium levels are restored to a level above about 3.7 mEq/IU per liter.

11. A method of restoring serum magnesium levels, comprising the step of administering a highly bioavailable magnesium salt in a pharmaceutically acceptable amount sufficient to restore depleted serum magnesium levels to a therapeutically acceptable level.

12. The method of claim 11, wherein the highly bioavailable magnesium salt has a bioavailability of at least about 30%.

13. The method of claim 12, wherein the highly bioavailable magnesium salt has a bioavailability of at least about 40%.

14. The method of claim 13, wherein the highly bioavailable magnesium salt comprises magnesium l-lactate dihydrate.

15. The method of claim 11, wherein the highly bioavailable magnesium salt is formulated in a formulation selected from a group consisting essentially of an immediate release tablet, a capsule, a gel, an ingestible liquid, a powder, a patch, and an intravenous injection.

16. The method of claim 11, wherein the highly bioavailable magnesium salt is formulated in a formulation selected from a group consisting essentially of a sustained release tablet, a capsule, a gel, an ingestible liquid, a powder, a patch, and an intravenous injection.

17. The method of claim 11, wherein the highly bioavailable magnesium salt is administered in a dosage of between about 3 mEq/IU and about 60 mEq/IU per day.

18. The method of claim 17, wherein the highly bioavailable magnesium salt is administered in a dosage of about 40 mEq/IU per day.

19. The method of claim 11, wherein depleted serum potassium levels are restored to a level above about 3.7 mEq/IU per liter.

20. A prescription dispensing system for treating renal magnesium wasting comprising:
(a) a first pharmaceutically acceptable dosage unit of a highly bioavailable magnesium salt; and
(b) a second pharmaceutically acceptable dosage unit of a drug, a known side effect of which is renal magnesium wasting.

21. The prescription dispensing system of claim 20, wherein the highly bioavailable magnesium salt has a bioavailability of at least about 30%.

22. The prescription dispensing system of claim 21, wherein the highly bioavailable magnesium salt has a bioavailability of at least about 40%.

23. The prescription dispensing system of claim 22, wherein the highly bioavailable magnesium salt comprises magnesium l-lactate dihydrate.

24. The prescription dispensing system of claim 20, wherein the highly bioavailable magnesium salt is formulated in a formulation selected from a group consisting essentially of an immediate release tablet, a capsule, a gel, an ingestible liquid, a powder, a patch, and an intravenous injection.

25. The prescription dispensing system of claim 20, wherein the highly bioavailable magnesium salt is formulated in a formulation selected from a group consisting essentially of a sustained release tablet, a capsule, a gel, an ingestible liquid, a powder, a patch, and an intravenous injection.

26. The prescription dispensing system of claim 20, wherein the first pharmaceutically effective dosage unit is between about 3 mEq/IU and about 60 mEq/IU.

27. The prescription dispensing system of claim 20, wherein the renal magnesium wasting medication is selected from a group consisting essentially of a diuretic, an immunosuppressant, and a chemotherapeutic agent.

28. The prescription dispensing system of claim 20, wherein the first pharmaceutically effective dosage unit is selected from a group consisting essentially of a single dose, a daily regimen, and a multiple day regimen.

29. The prescription dispensing system of claim 20, wherein the second pharmaceutically effective dosage unit is selected from a group consisting essentially of a single dose, a daily regimen, and a multiple day regimen.

30. The prescription dispensing system of claim 20, further comprising at least one dispensing container pre-filled with said first pharmaceutically effective dosage unit and said second pharmaceutically effective dosage unit.

31. The prescription dispensing system of claim 30, wherein the at least one dispensing container is selected from a group consisting essentially of at least one blister pack, at least one bottle, and at least one syringe.

32. The prescription dispensing system of claim 20, wherein the first pharmaceutically effective dosage unit and the second pharmaceutically effective dosage unit are co-formulated prior to packaging.

33. The prescription dispensing system of claim 32, wherein the first pharmaceutically effective dosage unit and the second pharmaceutically effective dosage unit are co-formulated in a formulation selected from a group consisting essentially of a sustained release tablet, a capsule, a gel, an ingestible liquid, a powder, a patch and an intravenous injection.

34. The prescription dispensing system of claim 32, wherein the first pharmaceutically effective dosage unit and the second pharmaceutically effective dosage unit are co-formulated in a formulation selected from a group consisting essentially of a sustained release tablet, a capsule, a gel, an ingestible liquid, a powder, a patch and an intravenous injection.

35. A method for providing a renal magnesium wasting treatment regimen, comprising the steps of:
(a) formulating a renal magnesium wasting treatment regimen comprising a pharmaceutically acceptable dosage unit of a highly bioavailable magnesium salt and a pharmaceutically acceptable dosage unit of a renal magnesium wasting drug; and
(b) filling at least one first dispensing container with the pharmaceutically acceptable dosage unit of the highly bioavailable magnesium salt and the pharmaceutically acceptable dosage unit of the renal magnesium wasting drug.

36. The method of claim 35, wherein said highly bioavailable magnesium salt has a bioavailability of at least about 30%.

37. The method of claim 36, wherein said highly bioavailable magnesium salt has a bioavailability of at least about 40%.

38. The method of claim 37, wherein said highly bioavailable magnesium salt comprises magnesium l-lactate dihydrate.

39. The method of claim 35, wherein said highly bioavailable magnesium salt is formulated in a formulation selected from a group consisting essentially of a sustained release capsule, a tablet, a gel, an ingestible liquid, a powder, a patch and an intravenous injection.

40. The method of claim 35, wherein said highly bioavailable magnesium salt is formulated in a formulation selected from a group consisting essentially of an immediate release capsule, a tablet, a gel, an ingestible liquid, a powder, a patch and an intravenous injection.

41. The method of claim 35, wherein the first pharmaceutically effective dosage unit is between about 3 mEq/IU and about 60 mEq/IU.

42. The method of claim 35, wherein the renal magnesium wasting drug is selected from a group consisting essentially of a diuretic, an immunosuppressant, and a chemotherapeutic agent.

43. The method of claim 35, wherein the first pharmaceutically effective dosage unit is selected from a group consisting essentially of a single dose, a daily regimen, and a multiple day regimen.

44. The method of claim 35, wherein the second pharmaceutically effective dosage unit is selected from a group consisting essentially of a single dose, a daily regimen, and a multiple day regimen.

45. The method of claim 35, wherein the dispensing container is selected from a group consisting essentially of at least one blister pack, at least one bottle, and at least one syringe.

46. The method of claim 35, further comprising the step of co-formulating the first pharmaceutically effective dosage unit and the second pharmaceutically effective dosage unit prior to packaging.

47. The method of claim 46, wherein the first pharmaceutically effective dosage unit and the second pharmaceutically effective dosage unit are co-formulated in a formulation selected from a group consisting essentially of a sustained release tablet, a capsule, a gel, an ingestible liquid, a powder, a patch and an intravenous injection.

48. The method of claim 46, wherein the first pharmaceutically effective dosage unit and the second pharmaceutically effective dosage unit are co-formulated in a formulation selected from a group consisting essentially of a sustained release tablet, a capsule, a gel, an ingestible liquid, a powder, a patch and an intravenous injection.
